(19) United States
(12) Patent Application Publication
(10) Pub. No.: US 2025/0009358 A1
(43) Pub. Date: Jan. 9, 2025

(54) VASCULAR OCCLUSION DEVICES AND METHODS FOR OCCLUDING A VESSEL

(71) Applicant: Bard Peripheral Vascular, Inc., Franklin Lakes, NJ (US)

(72) Inventors: Emily A. Goel, Phoenix, AZ (US); Logan J. Grace, Laveen, AZ (US); Michaella Kavanagh, Minneapolis, MN (US); Oleg Stanilevskiy, Chandler, AZ (US); Jeffrey Wang, Phoenix, AZ (US)

(73) Assignee: Bard Peripheral Vascular, Inc., Franklin Lakes, NJ (US)

(21) Appl. No.: 18/702,090

(22) PCT Filed: Oct. 21, 2021
(86) PCT No.: PCT/US2021/056012

§ 371 Date: Apr. 17, 2024

(51) Int. Cl. A61B 17/12; A61B 17/00

(52) U.S. Cl. CPC A61B 17/12145; A61B 17/12109; A61B 17/1215; A61B 2017/00867; A61B 2017/12054

(57) ABSTRACT

A vascular occlusion device includes a plurality of radially-extending segments and a plurality of angled bends connecting ends of two or more radially-extending segments of the plurality of radially-extending segments. Each angled bend may define a contact point configured to anchor to a vessel wall. The vascular occlusion device may be configurable between an unexpanded state and an expanded state, and when the vascular occlusion device is in the expanded state, the plurality of angled bends expand such that the contact points contact the vessel wall. The contact points may define a width of the vascular occlusion device in a direction perpendicular to a length of the vascular occlusion device. The width may be configured to correspond to a diameter of a blood vessel.

### TECHNICAL FIELD

The present specification generally relates to vascular occlusion devices and methods for occluding a vessel.

### BACKGROUND

Vascular occlusion or embolization devices are intravascular implants that are intended to occlude blood flow in percutaneous interventions. For example, a vascular occlusion device may be positioned to control hemorrhaging due to aneurysms, certain tumors, and arteriovenous malformations. Vascular occlusion devices may also be positioned to block blood vessels providing flow to certain types of tumors. Existing embolization devices may include helically-wound coils extending lengthwise through a portion of a vessel. Such helical coil-based structures may lack structural support elements extending in a radial direction. As a result, existing coil-based devices may be at risk of migration or other unwanted movement.

### SUMMARY

Embodiments of the present disclosure are directed to improvements over the above limitations by providing vascular occlusion devices that occlude vessels using zig-zag shaped structural elements. The zig-zagged shaped structural elements may provide more radial force against the vessel walls than coil-based devices and therefore reduce the risk of migration thereof within the vessel.

According to one embodiment of the present disclosure, a vascular occlusion device includes a plurality of radially-extending segments and a plurality of angled bends connecting ends of two or more radially-extending segments of the plurality of radially-extending segments. Each angled bend may define a contact point configured to anchor to a vessel wall. The vascular occlusion device may be configurable between an unexpanded state and an expanded state, and when the vascular occlusion device is in the expanded state, the plurality of angled bends expand such that the contact points contact the vessel wall. The contact points may define a width of the vascular occlusion device in a direction perpendicular to a length of the vascular occlusion device. The width may be configured to correspond to a diameter of a blood vessel.

According to another embodiment of the present disclosure, a vascular occlusion device for occluding a vessel via contact with a vessel wall includes a plurality of radially-extending segments and a plurality of angled bends connecting ends of two or more radially-extending segments of the plurality of radially-extending segments. Each angled bend may define a contact point configured to anchor to the vessel wall. The vascular occlusion device is configurable between an unexpanded state and an expanded state, and when the vascular occlusion device is in the expanded state, the plurality of angled bends expand such that the contact points contact the vessel wall. The plurality of radially-extending segments may extend radially between successive contact points to occlude blood flow in the vessel.

According to yet another embodiment of the present disclosure, a method of occluding a blood vessel includes positioning a vascular occlusion device at a desired occlusion position within a blood vessel. The method also includes expanding the vascular occlusion device into an expanded state such that contact points of a plurality of angled bends connecting a plurality of radially-extending segments of the vascular occlusion device contact walls of the blood vessel. The contact points define a width of the vascular occlusion device in a direction perpendicular to a length of the vascular occlusion device, and the width is configured to correspond to a diameter of a blood vessel.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a vascular occlusion device in an unexpanded state as delivered to a vessel by a catheter assembly, according to one or more embodiments described herein;

FIG. 1B schematically depicts the vascular occlusion device of FIG. 1B in an expanded state to occlude blood flow within the vessel, according to one or more embodiments described herein;

FIG. 1C schematically depicts a cross-sectional view of the vessel and vascular occlusion device through the line 1C-1C of FIG. 1B, according to one or more embodiments described herein;

FIG. 2A schematically depicts a plurality of vascular occlusion devices in unexpanded states as delivered to a vessel by a catheter assembly, according to one or more embodiments described herein;

FIG. 2B schematically depicts the plurality of vascular occlusion devices in an expanded stated to occlude blood flow within the vessel, according to one or more embodiments described herein;

FIG. 2C schematically depicts an axial end view of the plurality of vascular occlusion devices depicted in in FIGS. 2A and 2B in the expanded state, according to one or more embodiments described herein;

FIG. 3 schematically depicts a vascular occlusion device in an expanded state and including a detachment mechanism disposed in a blood vessel, according to one or more embodiments described herein;

FIG. 4 schematically depicts a vascular occlusion device in an expanded state and including surface treatments at a plurality of angled bends thereof, according to one or more embodiments described herein; and

FIG. 5 depicts a flow diagram of a method of occluding a blood vessel using an occlusion device with a zig-zag structure, according to one or more embodiments described herein.

### DETAILED DESCRIPTION

Embodiments of the present disclosure are directed to vascular occlusion devices including zig-zag-shaped structural components including a plurality of radially-extending segments and a plurality of angled bends connecting ends of two or more of the radially-extending segments. Each angled bend may define a contact point configured to anchor to a vessel wall. When deployed, the vascular occlusion device may expand from an unexpanded state to an expanded state such that that the contact points contact the vessel wall. The contact points may define a width of the vascular occlusion device in a direction perpendicular to a length of the vascular occlusion device, and the width may be configured to correspond to a diameter of the blood vessel. As a result, at least a portion of the plurality of radially-extending segments may extend radially and/or longitudinally through the vessel thereby at least partially occluding blood flow within the vessel. For example, the radially extending segments may be positioned at some oblique angle relative to a centerline of a blood vessel. The occlusion devices of the present disclosure may serve as an alternative to coil-based devices. As such, the occlusion devices described herein may either be pushable and/or detachable, such that the occlusion devices may be removably attached to a delivery device (e.g., wire, rod, or the like) and retrieved via the delivery device or another suitable retrieval tool. Beneficially, the radially-extending segments of the vascular occlusion device may compress the angled bends into the walls of the vessel, thereby lowering the probability of device migration over coil-based devices.

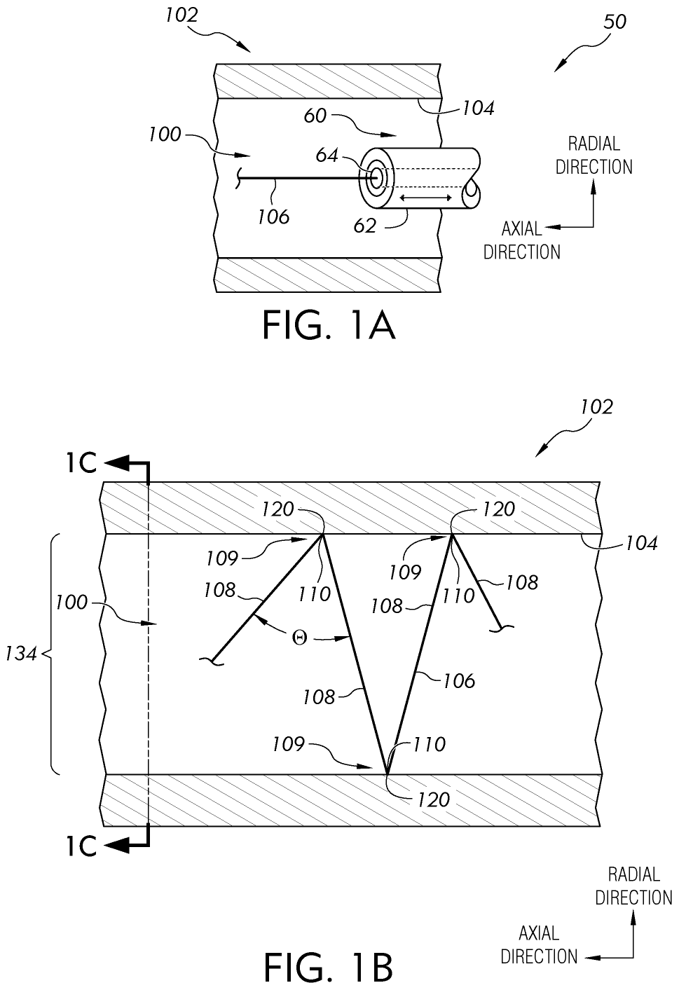

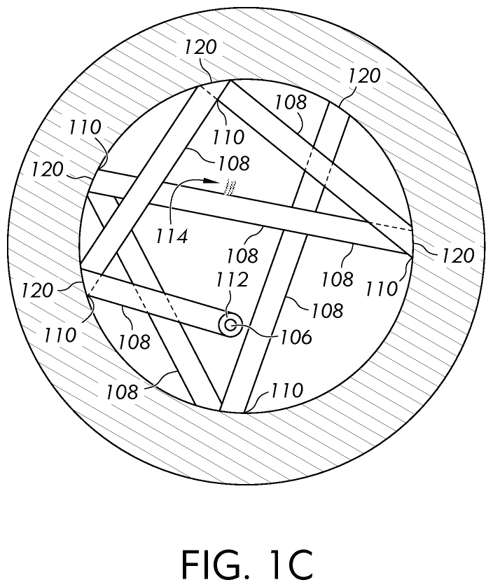

FIGS. 1A and 1B schematically depict a system 50 for occluding a blood vessel 102. For example, the blood vessel 102 may include any artery, vein, capillary, or the like. The system 50 generally includes a catheter assembly 60 and a vascular occlusion device 100. FIG. 1A schematically depicts the vascular occlusion device 100 in an unexpanded state as it is delivered to a desired occlusion position within the blood vessel 102 via the catheter assembly 60. FIG. 1B schematically depicts the vascular occlusion device 100 in an expanded state after deployment via the catheter assembly 60. As shown in FIG. 1A, the catheter assembly 60 comprises a catheter 62 and a pusher element or delivery wire 64 contained in the catheter 62. The vascular occlusion device 100 generally includes a shape memory body 106 formed of a suitable shape memory material (e.g., Nitinol or a suitable shape memory polymer such as some polyurethanes and polyethylene terephthalate) and is compressed into the depicted unexpanded shape and inserted into the catheter 62. In embodiments, the vascular occlusion device 100 may be delivered from the catheter assembly 60 into the blood vessel 102 by actuating or moving the pusher element 64 such that the pusher element 64 pushes the vascular occlusion device 100 out of the catheter 62. In embodiments, multiple vascular occlusion devices 100 can be delivered via a single catheter assembly 60. For example, vascular occlusion devices 100 (e.g., two or more, four or more, six or more, eight or more, ten or more, etc.) may be positioned sequentially within a catheter assembly 60, and may be sequentially deployed until the desired number of vascular occlusion devices 100 and/or the desired amount of occlusion is achieved. In embodiments, each vascular occlusion device 100 may have an unexpanded length of between about 20 and about 100 mm, such as about 50 mm, though other unexpanded lengths are contemplated and possible. It is noted that while the occlusion device 100 is shown as being delivered in an unexpanded state to the blood vessel 102, the occlusion device 100 may transition from the unexpanded state to the expanded state as it is being delivered from the catheter 62 to the blood vessel 102. Accordingly, the occlusion device 100 may begin expanding as it exits the catheter 62.

As shown in FIG. 1B, when the vascular occlusion device 100 is in an expanded state, the shape memory body 106 may generally include a plurality of radially-extending segments 108 coupled to one another via a plurality of bending regions 109, which extend at least partly in a radial direction of the vessel but may additionally extend partly in a longitudinal direction of the vessel. In embodiments, the plurality of radially-extending segments 108 are integrally formed with the plurality of bending regions 109 (e.g., the shape memory body 106 may be formed of a single piece or wire of shape memory material). In embodiments, the plurality of radially-extending segments 108 are reinforced with wire wound around the shape memory material. In embodiments, the plurality of radially-extending segments 108 and the plurality of bending regions 100 are separately formed and coupled to one another via any conventional joining technique (e.g., brazing, welding, adhesives, or the like). The shape memory material of the shape memory body 106 may be thermally treated to cause expansion of the vascular occlusion device 100 into the expanded state depicted in FIG. 1B when heated to a desired temperature (e.g., body temperature) after deployment (e.g., via the catheter assembly 60).

As depicted in FIG. 1B, once in the expanded shape, the vascular occlusion device 100 forms a zig-zag structure including a plurality of radially-extending segments 108 and a plurality of angled bends 110 formed within the bending regions 109 and connecting ends of the plurality of radially-extending segments 108 to one another. The shape memory body 106 may extend radially within the blood vessel 102 between different portions of the wall 104 such that the plurality of angled bends 110 define contact points 120 with the portions of the wall 104. The vascular occlusion device 100 may compress in a longitudinal direction such that the plurality of radially-extending segments 108 extend and the plurality of angled bends 110 are driven into the wall 104, thereby anchoring the vascular occlusion device 100 at a desired occlusion position.

In embodiments, the vascular occlusion device 100 self-expands into the expanded state depicted in FIG. 1B via the shape memory effect. For example, when fabricated, the vascular occlusion device 100 may be manipulated in shape to comprise a width 134 that is greater than or equal to a diameter of the blood vessel 102, if allowed to expand unimpeded. When in the blood vessel 102, the vascular occlusion device 100 may expand until stopped by the wall 104 of the blood vessel 102. As a result of the mechanical impedance from the wall 104, the plurality of radially-extending segments 108 may apply a radial force against the wall 104 via the plurality of angled bends 110. The radial force supplied by the plurality of radially-extending segments 108 may anchor the plurality of angled bends 110 into the wall 104, thereby securing the vascular occlusion device 100 at a desired occlusion position. While the depicted plurality of radially-extending segments 108 are straight lines, it should be appreciated that the plurality of radially-extending segments 108 may be bent or curved (e.g., as a result of compression from encountering the wall 104) in any shape in accordance with the present disclosure. Embodiments are also envisioned where the plurality of radially-extending segments 108 do not extend in straight lines by design (e.g., some of the plurality of radially-extending segments 108 may include sub-segments that extend in different directions from one another) to facilitate occluding blood flow.

As shown in FIG. 1B, successive ones of the plurality of radially-extending segments 108 may extend at an angle θ relative to one another as a result of the plurality of angled bends 110. In embodiments, at least some of the plurality of plurality of radially-extending segments 108 include lengths that are greater than or equal to the diameter, D, of the blood vessel 102. Such a configuration may ensure that at least a portion of the plurality of radially-extending segments 108 are capable of extending through a geometric center of the blood vessel and supporting two or more the plurality of angled bends 110 contacting two opposing portions of the wall 104 of the blood vessel 102.

While the vascular occlusion device 100 is depicted to only include four radially-extending segments and three angled bends, any number of angled bends and radially-extending segments may be included consistent with the present disclosure. In embodiments, for example, the vascular occlusion device 100 includes at least 5 (e.g., at least 5, at least 6, at least 7, at least 10, at least 15, at least 20) angled bends, and in such embodiments, includes a number of radially-extending segments that is at least as high as the number of angled bends. Greater numbers of angled bends may beneficially provide a greater number of points of contact between the vascular occlusion device 100 and the blood vessel 102, thereby reducing the probability of device migration. A greater number of radially-extending segments may also aid in occluding blood flow within the blood vessel 102, as the radially-extending segments may extend in different directions within the blood vessel (e.g., at different azimuthal orientations relative to the depicted radial direction in the coordinate axis depicted in FIG. 1B). Increasing the number of radially-extending segments may increase the portion of the blood vessel 102 that is directly blocked by the vascular occlusion device 100, thereby providing more complete occlusion of blood flow.

In embodiments, the plurality of radially-extending segments 108 may have lengths that differ from one another. For example, in some embodiments, core ones of the radially-extending segments 108 (e.g., ones of the plurality of radially-extending segments 108 not disposed at an end of the vascular occlusion device 100) vary from one another in length. In such embodiments, angled bends extending from such a shorter linear segment may not contact the wall 104. Such angled bends may be suspended in the lumen of the blood vessel 102, but still aid in occluding blood flow. In some embodiments, less than all of the plurality of angled bends 110 contact the wall 104. A wide variety of combinations of radially-extending segment lengths, angles θ of the plurality of angled bends 110 and numbers of radially-extending segments and angled bends are contemplated and within the scope of the present disclosure.

In embodiments, the plurality of angled bends 110 comprise bent portions of shape memory material connecting successive ones of the plurality of radially-extending segments 108 to one another. In embodiments, the plurality of angled bends 110 extend along arc lengths corresponding to the angle θ that adjacent ones of the plurality of radially-extending segments 108 extend relative to one another. In embodiments, the plurality of angled bends 110 comprise more than one curved section of shape memory material such that they deviate in shape from a circular arc. For example, in some embodiments, at least one of the plurality of angled bends 110 include a linear section extending between portions of the vascular occlusion device 100 that are bent at the ends of the plurality radially-extending segments 108. Such a linear portion may extend parallel or substantially parallel (e.g., within 5° of parallel) to the wall 104. Such linear portions may beneficially increase the contact areas between the plurality of angled bends 110 and the wall 104 and stabilize the vascular occlusion device 100. In embodiments, at least one of the plurality of angled bends 110 includes an m-shaped section of shape memory material such that those angled bends establish multiple points of contact between the wall 104 and the vascular occlusion device 100. In embodiments, at least one of the plurality of angled bends 110 comprises a substantially v-shaped section of shape memory material to establish a single point of contact with the wall 104. Such a configuration may concentrate the radial force supplied by the plurality of radially-extending segments 108 and result in a more secure anchoring of the vascular occlusion device to the wall 104.

In embodiments, the vascular occlusion device 100 is shaped such that the angle θ that the plurality of radially-extending segments 108 extend relative to one another varies as a function of lengthwise position on the vascular occlusion device 100. Such a configuration may result in the points of contact between the vascular occlusion device 100 and the wall 104 being non-uniformly distributed as a function of length. In embodiments, the radial force applied to the wall 104 by a particular angled bend 110 may vary in inverse proportion to the value of the angle θ at which the shape memory body 106 is bent within that angled bend 110. As such, the magnitude of the angles by which the vascular occlusion device 100 is bent at each of the plurality of angled bends 110 may be used to tune a distribution of radial force applied by the vascular occlusion device 100 to the wall 104. In embodiments, the angle θ decreases as a function of increasing distance from a lengthwise center of the vascular occlusion device 100 such that the radial force is greatest at the angled bends disposed proximate to the ends of the vascular occlusion device 100. Such a configuration may result in the ends of the vascular occlusion device 100 being anchored, while allowing inner (e.g., between either longitudinal end) radially-extending segments to be longer to occlude greater portions of the blood vessel 102. In embodiments, the angle θ is a maximum value at a center of the vascular occlusion device 100 (e.g., such that radially-extending segments closest to a lengthwise center of the vascular occlusion device 100 extend closer to parallel to the wall 104 than those at the ends of the vascular occlusion device 100) to provide the ends of the vascular occlusion device 100 flexibility to engage the wall 104 at a variety of different locations.

Embodiments are also envisioned where the vascular occlusion device 100 includes a branched structure where certain ones of plurality of radially-extending segments 108 extend outward from other ones of plurality of radially-extending segments 108. In embodiments, at least one of the plurality of radially-extending segments 108 may extend outward from another of the plurality of radially-extending segments 108 at a location other than one of the plurality of angled bends 110. Such an outwardly extending radially-extending segment may include a plurality of sub-segments that are connected to one another via a plurality of additional angular bends. That is, certain ones of the plurality of radially-extending segments 108 may substantially correspond in structure to the vascular occlusion device 100 when in the expanded state depicted in FIG. 1B, but branch outward from another linear segment. Such a configuration may facilitate forming a complex network of radially-extending segments extending in a plurality of different directions to occlude blood flow within the blood vessel 102.

FIG. 1C depicts down-axis cross-sectional view of the blood vessel 102 and vascular occlusion device 100, in accordance with an example embodiment of the present disclosure. As shown, the vascular occlusion device 100 is depicted to include a coating 112 disposed on the shape memory body 106 although in some embodiments the device 100 may be provided without a coating 112. In embodiments, the coating 112 covers an entirety of the shape memory body 106. In embodiments, the coating 112 covers only a portion of the shape memory body 106. For example, in embodiments, the coating 112 may be removed during surface treatments of the plurality of angled bends 110 (e.g., during surface roughening thereof). While the coating 112 is depicted to only include a single coating layer, embodiments are also envisioned where an intermediate coating (not depicted) is disposed between the shape memory body 106 and the coating 112. Such an intermediate coating may include an adhesive layer configured to improve the adhesion between the shape memory body 106 and the coating 112.

In embodiments, the coating 112 is a thrombogenic coating configured to increase thrombogenicity within blood flow proximate to the vascular occlusion device 100. The thrombogenic coating may include any suitable material with sufficient porosity to enhance thrombogenicity. In embodiments, the coating 112 comprises a woven network of polymeric fiber-based material. In embodiments, the coating 112 comprises a suitable thrombogenic agent. Examples of suitable thrombogenic agents may include, but are not limited to, a hydrophobic polymer (e.g. polyurethane), silver plating or deposition, gold plating or deposition, or the like. In embodiments the coating 112 comprises a positively charged surface treatment. For example, the coating 112 may include a suitable polymer-based material functionalized with ionic groups to induce a positive polarity on the surface of the vascular occlusion device 100 to increase thrombogenicity within the blood vessel 102 proximate the vascular occlusion device 100. Examples of using suitable polymer-based material functionalized with ionic groups to induce a positive polarity on the surface may include, but are not limited to, introducing oxygen rich groups via plasma treatment, e.g. PVDF (polyvinylidene fluoride) treated with argon (Ar) and oxygen (O2), applying positively charged polymers, such as poly(ethylenimine) and poly(l-lysine), or the like.

In embodiments, the coating 112 is a hydrogel coating configured to increase the surface area of the vascular occlusion device 100 and increase friction with the wall 104. The hydrogel may include a composition that expands upon heating to body temperature so as to increase a surface area of the vascular occlusion device 100 once deployed. The coating 112 may include any suitable biomaterial for providing any additional functionality to the vascular occlusion device 100. For example, the coating 112 may be configured to deliver one or more therapeutic agents or the like. In embodiments, the coating 112 is selected to provide desired surface properties (e.g., surface roughness, bonding capabilities, and the like) to at least a portion of the vascular occlusion device 100. In embodiments, the coating 112 includes a suitable metallic or polymeric-based material that may be subjected to further processing (e.g., grinding, machining) to induce such desired surface properties.

Referring still to FIG. 1C, in embodiments, as an alternative or in addition to the coating 112, the vascular occlusion device 100 may include thrombogenic fibers 114 extending from at least a portion the shape memory body 106. The thrombogenic fibers 114 may comprise a suitable polymeric material (e.g., polyethylene terephthalate (PET), polytetrafluoroethylene (PTFE), or other suitable material) that are adhered to the coating 112 or the shape memory body 106 to increase thrombogenicity proximate to the vascular occlusion device 100. The thrombogenic fibers 114 facilitate providing additional functionality via the coating 112 (e.g., increased surface area via a hydrogel), while still increasing thrombogenicity to promote occlusion.

As depicted in FIG. 1C, in embodiments, the plurality of angled bends 110 contact the wall 104 at different angular regions of the blood vessel 102. That is, the plurality of radially-extending segments 108 may not extend in the same plane as one another. As a result, the shape memory body 106 may include a twisted configuration when in the expanded state. Such a twisted configuration may result in the plurality of radially-extending segments 108 extending in different directions within the blood vessel 102 to form a network of linear structures to occlude blood flow within the blood vessel 102. In embodiments, such a twisted configuration may result from thermally treating the shape memory material of the vascular occlusion device 100. In embodiments, successive ones of the plurality of radially-extending segments 108 extend at different angles relative to an axial direction. The non-uniform angular distribution of the plurality of angled bends 110, resulting from the non-uniform extension directions of the plurality of radially-extending segments 108, facilitates the formation of a complex network of radially-extending segments along a blood flow direction, thereby occluding blood flow within the blood vessel 102.

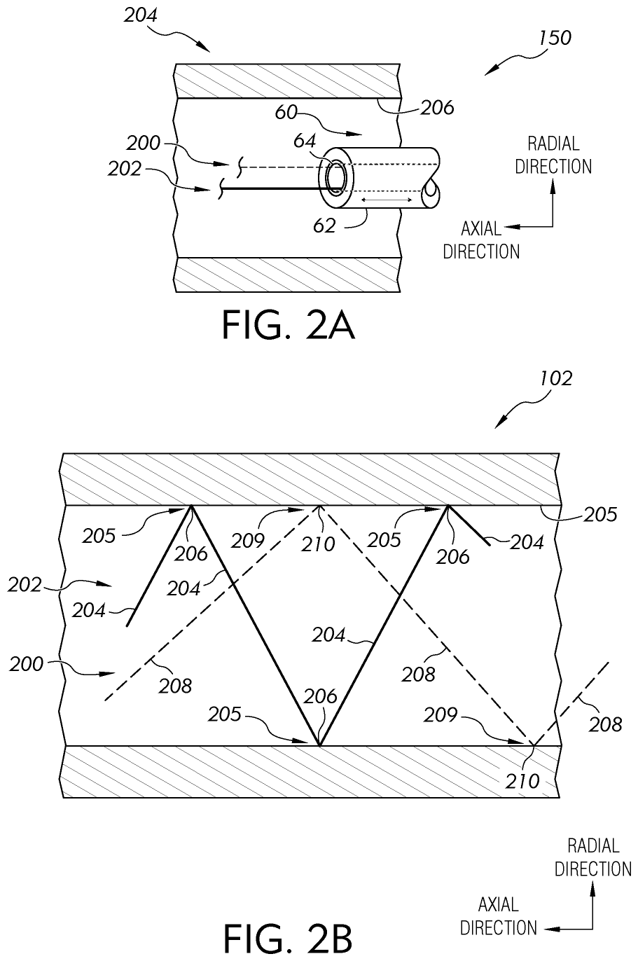

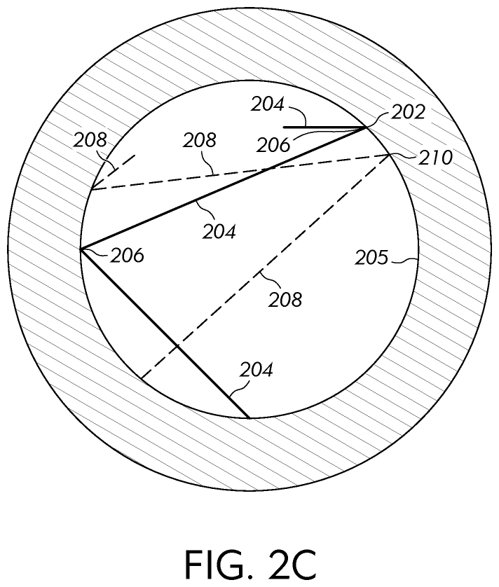

FIGS. 2A and 2B schematically depict another system 150 for occluding a blood vessel 204, according to an example embodiment. The system 150 may include the catheter assembly 60 described herein with respect to FIGS. 1A-1C, as well as a plurality of vascular occlusion devices 200 and 202. FIG. 2A schematically depicts the plurality of vascular occlusion device 200 and 202 in an unexpanded state as delivered to a desired occlusion position within a blood vessel 204 via the catheter assembly 60. In embodiments, each of the plurality of vascular occlusion devices 200 and 202 may be similar in structure to the vascular occlusion device 100 described herein with respect to FIGS. 1A-1C. As such, when in the unexpanded state, the plurality of vascular occlusion devices 200 and 202 may comprise substantially linear shaped wires of shape memory material or have another suitable form. The plurality of vascular occlusion devices 200 and 202 may be simultaneously delivered to a desired occlusion position within the blood vessel via the catheter 62 or may be delivered in series.

In embodiments, the plurality of vascular occlusion devices 200 and 202 are formed from a shape memory material and self-expand into the expanded state depicted in FIG. 2B. As shown, when in the expanded state, the vascular occlusion device 200 includes a plurality of radially-extending segments 208 that are connected to one another via one or more bending regions 209. The vascular occlusion device 202 also includes a plurality of radially-extending segments 204 that are connected to one another via a plurality of angled bends 205. In embodiments, the vascular occlusion devices 200 and 202 include shape memory bodies that are bent at the plurality of bending regions 205 and 209 to form pluralities of angled bends 206 and 210, respectively. Each of the plurality of vascular occlusion devices 200 and 202 may be similar in structure to the vascular occlusion device 100 described herein with respect to FIGS. 1A-1C when in an expanded state.

In embodiments, the plurality of vascular occlusion devices 200 and 202 may differ from one another in shape when expanded. For example, in the depicted embodiment, the radially-extending segments 208 of the vascular occlusion device 200 extend at a slightly greater angle relative to each other via the angled bends 210 as compared to the angle that the radially-extending segments 204 of the vascular occlusion device 202 extend relative to each other via the angled bend 206. Such a difference in structure may facilitate provision of a more dense network of points of contact with a wall 206 of the blood vessel 204 than possible if only a single vascular occlusion device were used. For example, in embodiments, the plurality of vascular occlusion devices 200 and 202 may each occupy non-overlapping points in space at each lengthwise position within the blood vessel 204 to form an interwoven structure of separate shape memory components. Such an interwoven structure may facilitate occluding the blood vessel 204.

In embodiments, the plurality of vascular occlusion devices 200 and 202 may contact one another within the blood vessel 204. For example, portions of the radially-extending segments of each of the plurality of vascular occlusion devices 200 and 202 may contact one another as the radially-extending segments traverse the interior of the blood vessel 204. Such contact may result in bending of the radially-extending segments in the radial direction, which may increase the occlusive propensity of the combination of the plurality of vascular occlusion devices 200 and 202 and also increase the anchoring force provided by the radially-extending segments by increasing the radial components of the compressive force stored in the radially-extending segments. In embodiments, the shapes of the plurality of vascular occlusion devices 200 and 202 in the expanded state depicted in FIG. 2B may be determined by thermal treatments thereof. That is, the plurality of vascular occlusion device 200 and 202 may each be configured to take a different shape. In embodiments, the plurality of vascular occlusion device 200 and 202 are identical, but differing orientations thereof resulting from deployment may result in the plurality of vascular occlusion device 200 and 202 contacting different portions of the wall 206.

FIG. 2C provides an axial view of the plurality of vascular occlusion devices 200 and 202 from a downstream portion of the blood vessel 204 (e.g., to the left of the depicted portion of the blood vessel 204 depicted in FIG. 2B). In embodiments, the radially-extending segments 204 and 208 of each of the plurality of vascular occlusion devices 200 and 202 may extend in different directions such that the angled bends of each of the plurality of vascular occlusion devices 200 and 202 contact different angular portions of the wall 206. As a result, even though the plurality of vascular occlusion devices 200 and 202 are wires having relatively small cross-sectional areas, the plurality of radially-extending segments may block an entirety of the blood vessel 204 in a direction perpendicular to the flow via a complex network of radially-extending segments extending in different directions.

In embodiments, any number of separate vascular occlusion devices similar in structure to the vascular occlusion device 100 described herein with respect to FIGS. 1A-1C may be used in combination to occlude blood flow within a blood vessel. Such vascular occlusion devices may be thermally treated to form a variety of different shapes when expanded to create a network of radially-extending segments extending radially in different regions of a blood vessel to occlude blood flow in the vessel. In embodiments, 2, 3, 4, 5, 6, 7, 8, 9, 10, or an even greater number of separate shape memory components configured to form zig-zag structures may be used in combination to occlude blood flow.

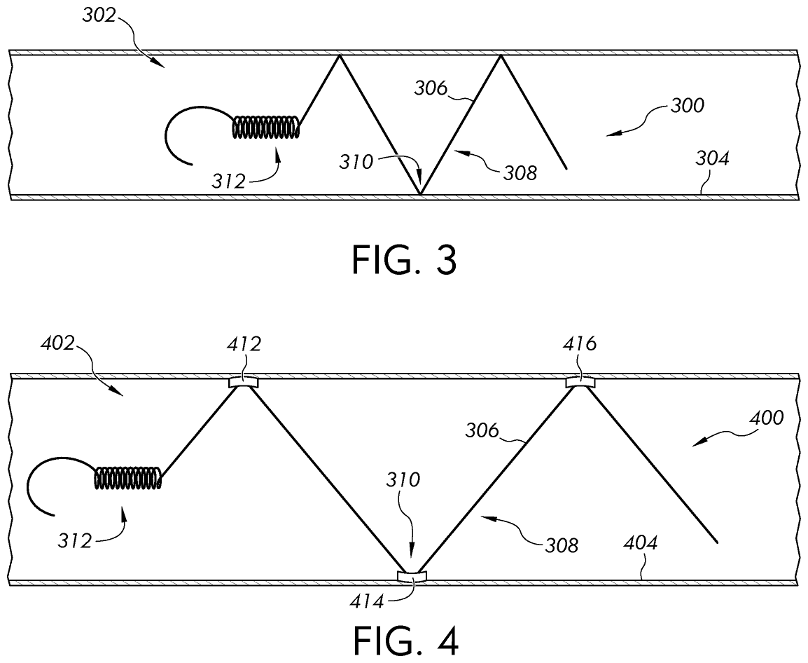

FIG. 3 schematically depicts a vascular occlusion device 300 in an expanded state and deployed within a blood vessel 302. In embodiments, the vascular occlusion device 300 may be substantially similar in structure to the vascular occlusion device 100 described herein with respect to FIGS. 1A-1C. For example, the vascular occlusion device 300 is depicted to include a shape memory body 306 including a plurality of radially-extending segments 308 that are interconnected with one another via a plurality of angled bends 310 that form a plurality of points of contact between the shape memory body 306 and a wall 304 of the blood vessel 302.

The vascular occlusion device 300 differs in structure from the vascular occlusion device 100 described herein in that the vascular occlusion device 300 is depicted to include a detachment mechanism 312. The detachment mechanism 312 may facilitate retrieval of the vascular occlusion device 300 using a suitable retrieval tool. In embodiments, the detachment mechanism 312 is formed of a different material than the shape memory body 306. For example, in embodiments, the detachment mechanism is formed of a suitable metallic material or alloy, such as stainless steel. Formation of the detachment mechanism from a different material than the shape memory body 306 may facilitate using machining techniques to shape the detachment mechanism 312 that are incompatible with shape memory material. In embodiments, the detachment mechanism 312 is formed of the same material as the shape memory body 306.

In the depicted embodiment, the detachment mechanism 312 is depicted to include a hook member to facilitate retrieval of the vascular occlusion device 300. The detachment mechanism 312 may be attached to the shape memory body 306 via a suitable attachment method (e.g., via a threaded connection, via adhesive). The detachment mechanism 312 may have a variety of forms depending on the implementation. For example, in embodiments, the detachment mechanism 312 includes a set of threads (or other suitable twisting element, such as a helical notch or protrusion) for attachment of a suitable threaded removal tool. In embodiments, the detachment mechanism 312 includes a magnetic element for engagement with a magnetic retrieval tool. Any suitable structure may be used for the detachment mechanism.

FIG. 4 schematically depicts a vascular occlusion device 400 in an expanded state and deployed within a blood vessel 402. The vascular occlusion device 400 may be substantially similar in structure to the vascular occlusion device 300 described herein with respect to and of FIGS. 1A-3. Accordingly, like references numerals are used in FIG. 4 to signify the incorporation of such like components. The device 400 may be provided with or without detachment mechanism 312. The vascular occlusion device 400 differs from the vascular occlusion device 300 described herein with respect to FIGS. 1A-3 in that the vascular occlusion device 400 includes surface treatments 412, 414, and 416 at the plurality of angled bends 310. The surface treatments 412, 414, and 416 may be configured to increase friction and/or surface area of the vascular occlusion device 400 at the plurality of angled bends 310 as compared to a vascular occlusion device lacking the surface treatments 412, 414, and 416. The surface treatments 412, 414, and 416 may comprise the same structure in some embodiments (e.g., such that the surface treatments 412, 414, and 416 are equivalent to one another). In embodiments, the surface treatments 412, 414, and 416 may differ from one another such that plurality of angled bends 310 may include different surface treatments.

In embodiments, the surface treatments 412, 414, and 416 comprise regions of the shape memory body 306 (or a coating disposed thereon) including a greater surface roughness than other portions of the shape memory body 306. In embodiments, the surface treatments 412, 414, and 416 may extend along the entire surface of the shape memory body 306. In embodiments, the surface treatments 412, 414, and 416 may comprise topographical modifications to have increased surface roughness along the entire surface of the shape memory body 306 to improve cell adhesion. In embodiments, the plurality of angled bends 310 include a surface roughness that is greater than that of the plurality of radially-extending segments 308. In some embodiments, the roughness parameter (Ra) is at least 10 nm, though other embodiments are contemplated and possible. For example, in some embodiments, the Ra is less than or equal to about 10,000 nm, though larger Ra's are contemplated and possible. The greater surface roughness at the plurality of angled bends 310 may increase friction with a wall 404 of the blood vessel 402 and thereby improve anchoring of the vascular occlusion device 400 and reducing the risk of migration. In embodiments, the increased roughness at plurality of angled bends 310 may be provided by surface finishing techniques (e.g., heat treatments, finishing, grinding, sanding, or the like) at predetermined positions on the shape memory body 306.

In embodiments, the surface treatments 412, 414, and 416 comprise additional sections of material applied to or added to the shape memory body 306 at the plurality of angled bends 310. For example, in embodiments, the surface treatments 412, 414, and 416 may include sections of a suitable polymeric material (e.g., PTFE) to facilitate bonding between the plurality of angled bends 310 and the wall 404. In embodiments, the surface treatments 414, 414, and 416 may include sections of metallic material (e.g., stainless steel or other suitable material) attached to the plurality of angled bends 310 (e.g., via welding, adhesive, or other suitable attachment mechanism). The additional material applied to the plurality of angled bends 310 may increase the contact area between the vascular occlusion device 400 and the wall 404, thereby improving anchoring. In embodiments, the additional materials applied to the plurality of angled bends 310 may be processed (e.g., via a suitable finishing or machine technique) to provide a surface roughness that is greater than that associated with the shape memory body 306 to enhance anchoring.

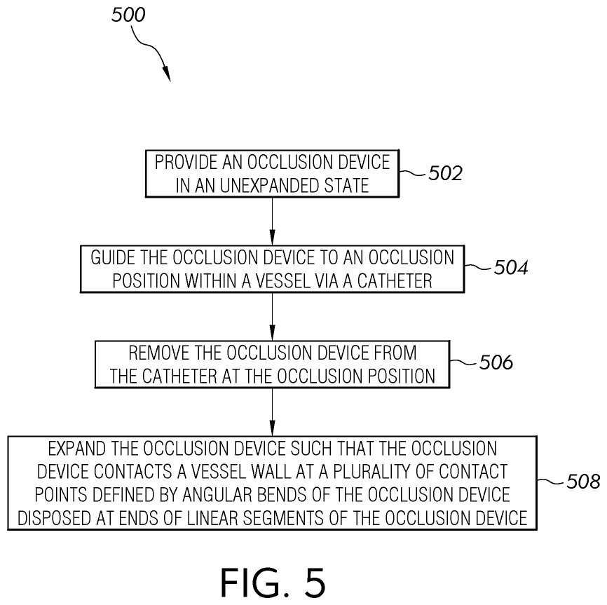

FIG. 5 depicts a flow diagram of a method 500 of occluding blood flow within a blood vessel using a vascular occlusion device. The method 500 may be performed, for example, to deploy the vascular occlusion device 100 described herein with respect to FIGS. 1A-1C to a desired occlusion position within the blood vessel 102 to occlude blood flow within the blood vessel 102. Accordingly, reference will be made to various components depicted in FIGS. 1A-1C to aid in the description of the method 500. In embodiments, the method 500 may be performed to deploy a plurality of vascular occlusion devices (such as the plurality of vascular occlusion devices 200 and 202 described herein with respect to FIGS. 2A-2C) into a blood vessel to occlude blood flow.

At block 502, the vascular occlusion device 100 is provided in an unexpanded state. For example, the shape memory body 106 described herein may be thermally treated at an elevated temperature to have a desired expanded shape, and, re-formed into the unexpanded state at a reduced temperature (e.g., beneath body temperature). The shape memory body 106 may be shaped to be a linear wire or a planar sheet (e.g. a ribbon) in the unexpanded state. At block 504, the vascular occlusion device 100 may be guided to an occlusion position within the blood vessel 102 using the catheter system 60. The catheter 62 may be a flexible tubing configured for traversal through the blood vessel 102 to the occlusion position. The catheter 62 may define a lumen into which the vascular occlusion device 100 and a suitable pusher element 64 are inserted. At block 506, the vascular occlusion device 100 is removed from the catheter 62 at the occlusion position using the pusher element 64.

At block 508, the vascular occlusion device 100 is expanded into the expanded state such that the vascular occlusion device 100 contacts the wall 104 at a plurality of contact points 120 defined by plurality of angled bends 110 disposed at ends of the plurality of radially-extending segments 108. As described herein, the plurality of radially-extending segments 108 may apply a radial force to the wall 104 via the plurality of angled bends 110. The radial force may anchor plurality of angled bends 110 into the wall 104, thereby preventing the vascular occlusion device 100 from migrating within the blood vessel 102. The vascular occlusion device 100 may be configured to take a complex shape such that the plurality of radially-extending segments 108 extend in a plurality of different directions to occlude blood flow, while still providing more radial force against the wall 104 than that typically provided by coil-based occlusion devices.

Depending on the design of the vascular occlusion device 100, the method 500 may include depositing additional vascular occlusion devices in addition to the vascular occlusion device 100 to occlude the blood flow. In embodiments, different vascular occlusion devices may be disposed at different axial locations within the blood vessel 102 to occlude blood flow. In embodiments, a plurality of vascular occlusion devices may be disposed at overlapping axial locations within the blood vessel 102 (see FIGS. 2A and 2B) to occlude blood flow. The method 500 may also occlude removing the vascular occlusion device 100, in embodiments where the vascular occlusion device 100 is designed to be detachable.

In embodiments, the vascular occlusion device 100 may be used in combination with other (e.g. coil-based) vascular occlusion devices. In embodiments, the vascular occlusion device 100 may serve as a filter to prevent an existing (e.g., already-deployed) occlusion device from migrating past a certain region within the blood vessel 102. In embodiments, the vascular occlusion device 100 may be deployed within a coil-based occlusion device to facilitate anchoring the coil-based occlusion device at a desired a position (e.g., the vascular occlusion device 100 may be delivered within a coil and expanded to contact the wall 104 in regions that don't overlap with the coil). As such, the vascular occlusion device 100 may be used in isolation to occlude blood flow, or in combination with any other occlusion device.

Embodiments can be described with reference to the following numerical clauses:
1. A vascular occlusion device configurable between an unexpanded state and an expanded state, the vascular occlusion device comprising: a plurality of radially-extending segments; and a plurality of bending portions that, in the expanded state, define angled bends connecting ends of two or more radially-extending segments of the plurality of radially-extending segments, wherein: each angled bend defines a contact point configured to anchor to a vessel wall, when the vascular occlusion device moves from the unexpanded state to the expanded state, the plurality of angled bends expand such that the contact points contact the vessel wall, the contact points define a width of the vascular occlusion device in a direction perpendicular to a length of the vascular occlusion device, and the width is configured to correspond to a diameter of a blood vessel.
2. The vascular occlusion device of any preceding clause, wherein: the vascular occlusion device comprises a single piece of shape memory material, and the vascular occlusion device self-transitions from the unexpanded state to the expanded state upon reaching a threshold temperature.
3. The vascular occlusion device of any preceding clause, wherein the shape memory material comprises nitinol.
4. The vascular occlusion device of any preceding clause, wherein, when the vascular occlusion device is in the unexpanded state, the vascular occlusion device comprises a wire or sheet of the shape memory material.
5. The vascular occlusion device of any preceding clause, wherein one or more radially-extending segments of the plurality of radially-extending segments extend radially through a geometric center of the vascular occlusion device.
6. The vascular occlusion device of any preceding clause, wherein the angled bends comprise an increased coefficient of friction as compared to the radially-extending segments.
7. The vascular occlusion device of any preceding clause, wherein the angled bends comprise a layer of polymeric material configured to contact the vessel wall.
8. The vascular occlusion device of any preceding clause, wherein the angled bends comprise a surface roughness that is greater than that of the radially-extending segments.
9. The vascular occlusion device of any preceding clause, wherein, when in the expended state, two angled bends disposed at opposite ends of a linear segment of the plurality of radially-extending segments are configured to contact different radial positions of a wall of the blood vessel.
10. The vascular occlusion device of any preceding clause, further comprising a hydrogel coating disposed on at least the angled bends.
11. The vascular occlusion device of any preceding clause, further comprising thrombogenic fibers extending from at least one of the radially-extending segments and the angled bends.
12. The vascular occlusion device of any preceding clause, further comprising a detachment mechanism extending from an end linear segment of the vascular occlusion device, the detachment mechanism being configured to engage with a removal tool for removing the vascular occlusion device from the blood vessel.
13. The vascular occlusion device of any preceding clause, wherein the detachment element comprises one or more of a twisting element, magnet, a hook, or a clamp hold.
14. A vascular occlusion device for occluding a vessel via contact with a vessel wall, the vascular occlusion device, configurable between an unexpanded state and an expanded state, the vascular occlusion device comprising: a plurality of radially-extending segments; and a plurality of bending portions that, in the expanded state, define angled bends connecting ends of two or more radially-extending segments of the plurality of radially-extending segments, wherein: each angled bend defines a contact point configured to anchor to a vessel wall, when the vascular occlusion device moves from the unexpanded state to the expanded state, the plurality of angled bends expand such that the contact points contact the vessel wall, and the plurality of radially-extending segments extend radially between successive contact points to occlude blood flow in the vessel.
15. The vascular occlusion device of any preceding clause, wherein: the vascular occlusion device comprises a single piece of shape memory material, and the vascular occlusion device self-transitions from the unexpanded state to the expanded state upon reaching a threshold temperature.
16. The vascular occlusion device of any preceding clause, wherein the shape memory material comprises nitinol.
17. The vascular occlusion device of any preceding clause, wherein the angled bends comprise an increased coefficient of friction as compared to the radially-extending segments.
18. The vascular occlusion device of any preceding clause, wherein: the angled bends comprise a layer of polymeric material configured to contact the vessel wall.
19. The vascular occlusion device of any preceding clause, wherein the angled bends comprise a surface roughness that is greater than that of the radially-extending segments.
20. The vascular occlusion device of any preceding clause, further comprising a hydrogel coating disposed on at least the angled bends.
21. The vascular occlusion device of any preceding clause, further comprising thrombogenic fibers extending from at least one of the radially-extending segments and the angled bends.
22. A method of occluding a blood vessel, the method comprising: positioning a vascular occlusion device at a desired occlusion position within a blood vessel, wherein the vascular occlusion device comprises: a plurality of radially-extending segments; and a plurality of bending portions that, in the expanded state, define angled bends connecting ends of two or more radially-extending segments of the plurality of radially-extending segments; and expanding the vascular occlusion device into an expanded state such that contact points of a plurality of angled bends connecting the plurality of radially-extending segments of the vascular occlusion device contact walls of the blood vessel, wherein: the contact points define a width of the vascular occlusion device in a direction perpendicular to a length of the vascular occlusion device, and the width is configured to correspond to a diameter of a blood vessel.
23. The method of any preceding clause, further comprising: positioning a plurality of vascular occlusion devices to the desired occlusion position; and expanding the plurality of vascular occlusion devices such that angled bends connecting a plurality of radially-extending segments of each vascular occlusion device contact the walls of the blood vessel.

It should now be understood that embodiments of the present disclosure are directed to vascular occlusion devices that occlude blood flow within a vessel via two or more radially-extending segments, with each of the two or more radially-extending segments having one or more angled bends disposed at an end thereof. The angled bends serve as contact points with the vessel wall, and the radially-extending segments radially force the angled bends against the vessel wall. Such radial force applied to the contact points via the radially-extending segments may reduce the migration risk of the vascular occlusion devices described herein as compared with coil-based occlusion devices. As a result, occlusion of undesired locations within the vessel may be avoided, thereby improving the safety of occlusion.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A vascular occlusion device configurable between an unexpanded state and an expanded state, the vascular occlusion device comprising:
a plurality of radially-extending segments; and
a plurality of bending portions that, in the expanded state, define angled bends connecting ends of two or more radially-extending segments of the plurality of radially-extending segments, wherein:
each angled bend defines a contact point configured to anchor to a vessel wall,
when the vascular occlusion device moves from the unexpanded state to the expanded state, the plurality of angled bends expand such that the contact points contact the vessel wall,
the contact points define a width of the vascular occlusion device in a direction perpendicular to a length of the vascular occlusion device, and
the width is configured to correspond to a diameter of a blood vessel.

2. The vascular occlusion device of claim 1, wherein:
the vascular occlusion device comprises a single piece of shape memory material, and
the vascular occlusion device self-transitions from the unexpanded state to the expanded state upon reaching a threshold temperature.

3. The vascular occlusion device of claim 2, wherein the shape memory material comprises nitinol.

4. The vascular occlusion device of claim 2, wherein, when the vascular occlusion device is in the unexpanded state, the vascular occlusion device comprises a wire or ribbon of the shape memory material.

5. The vascular occlusion device of claim 1, wherein in the expanded state, one or more radially-extending segments of the plurality of radially-extending segments are configured to extend radially through a geometric center of the vascular occlusion device.

6. The vascular occlusion device of claim 1, wherein the angled bends comprise an increased coefficient of friction as compared to the radially-extending segments.

7. The vascular occlusion device of claim 6, wherein the angled bends comprise a layer of polymeric material configured to contact the vessel wall.

8. The vascular occlusion device of claim 1, wherein the angled bends comprise a surface roughness that is greater than that of the radially-extending segments.

9. The vascular occlusion device of claim 1, wherein, when in the expanded state, two angled bends disposed at opposite ends of a linear segment of the plurality of radially-extending segments are configured to contact different radial positions of a wall of the blood vessel.

10. The vascular occlusion device of claim 1, further comprising a hydrogel coating disposed on at least the angled bends.

11. The vascular occlusion device of claim 1, further comprising thrombogenic fibers extending from at least one of the radially-extending segments and/or the angled bends.

12. The vascular occlusion device of claim 1, further comprising a detachment mechanism extending from an end radially-extending segment of the vascular occlusion device, the detachment mechanism being configured to engage with a removal tool for removing the vascular occlusion device from the blood vessel.

13. The vascular occlusion device of claim 12, wherein the detachment element comprises one or more of a twisting element, magnet, a hook, or a clamp hold.

14. A vascular occlusion device for occluding a vessel via contact with a vessel wall, the vascular occlusion device, configurable between an unexpanded state and an expanded state, the vascular occlusion device comprising:
a plurality of radially-extending segments; and
a plurality of bending portions that, in the expanded state, define angled bends connecting ends of two or more radially-extending segments of the plurality of radially-extending segments, wherein:
each angled bend defines a contact point configured to anchor to a vessel wall,
when the vascular occlusion device moves from the unexpanded state to the expanded state, the plurality of angled bends expand such that the contact points contact the vessel wall, and
the plurality of radially-extending segments extend radially between successive contact points to occlude blood flow in the vessel.

15. The vascular occlusion device of claim 14, wherein:
the vascular occlusion device comprises a single piece of shape memory material, and
the vascular occlusion device self-transitions from the unexpanded state to the expanded state upon reaching a threshold temperature.

16. The vascular occlusion device of claim 15, wherein the shape memory material comprises nitinol.

17. The vascular occlusion device of claim 14, wherein the angled bends comprise an increased coefficient of friction as compared to the radially-extending segments.

18. The vascular occlusion device of claim 17, wherein the angled bends comprise a layer of polymeric material configured to contact the vessel wall.

19. The vascular occlusion device of claim 14, wherein the angled bends comprise a surface roughness that is greater than that of the radially-extending segments.

20. The vascular occlusion device of claim 14, further comprising a hydrogel coating disposed on at least the angled bends.

21. The vascular occlusion device of claim 14, further comprising thrombogenic fibers extending from at least one of the radially-extending segments and/or the angled bends.

22. A method of occluding a blood vessel, the method comprising:
positioning a vascular occlusion device at a desired occlusion position within a blood vessel, wherein the vascular occlusion device comprises:
a plurality of radially-extending segments; and
a plurality of bending portions that, in the expanded state, define angled bends connecting ends of two or more radially-extending segments of the plurality of radially-extending segments; and
expanding the vascular occlusion device into an expanded state such that contact points of a plurality of angled bends connecting a plurality of radially-extending segments of the vascular occlusion device contact walls of the blood vessel, wherein:
the contact points define a width of the vascular occlusion device in a direction perpendicular to a length of the vascular occlusion device, and
the width is configured to correspond to a diameter of a blood vessel.

23. The method of claim 22, further comprising:
positioning a plurality of vascular occlusion devices to the desired occlusion position; and
expanding the plurality of vascular occlusion devices such that angled bends connecting a plurality of radially-extending segments of each vascular occlusion device contact the walls of the blood vessel.
